(19) United States
(12) Patent Application Publication
(10) Pub. No.: US 2018/0193609 A1
(43) Pub. Date: Jul. 12, 2018

(54) INFLATABLE BALLOON

(71) Applicant: The Provost, Fellows, Foundation Scholars, and the other members of the Board, of the College of the, Dublin (IE)

(72) Inventors: Catriona Lally, Dublin (IE); Owen Clarkin, Wexford (IE); James King, Cork (IE)

(21) Appl. No.: 15/821,898

(22) Filed: Nov. 24, 2017

Related U.S. Application Data
(63) Continuation of application No. PCT/EP2016/061943, filed on May 26, 2016.

(30) Foreign Application Priority Data
May 27, 2015 (GB) 1509065.7

(51) Int. Cl. A61M 25/10; A61F 2/24; A61F 2/958; A61M 29/02

(52) U.S. Cl. CPC A61M 25/1002; A61M 25/104; A61F 2/2433; A61M 2025/1084; A61M 29/02; A61M 2025/1059; A61M 2025/1068; A61F 2/958

(57) ABSTRACT

An inflation balloon 1 comprising a compliant balloon wall 3 and a first 5 and second 10 set of non-compliant filamentous members embedded in the balloon wall. The filamentous members are arranged to be slack so as to allow inflation of the inflation balloon, but to restrain the balloon to an expanded diameter when all of the slack has been taken up by expansion of the balloon. The first filamentous members undergo controlled failure to expand the balloon to a second larger expanded size.

### FIELD OF THE INVENTION

The present invention relates to an inflatable balloon for use in medical interventions, for example surgical techniques. An inflatable balloon of the invention is suitable for use in medical procedures in animals for example in humans. For example such an inflatable balloon may be utilised within the vasculature. The present invention includes catheter balloons.

### BACKGROUND TO THE INVENTION

Inflatable balloons are well known for use in various medical procedures.

Typically the balloon has a deflated (non-working) configuration. Typically this configuration is utilised while the balloon is being moved to a target treatment site. For example such a configuration is typically utilised while a balloon is being advanced through the body, for example through the vasculature to a target treatment site. Once at the target treatment site then the balloon is typically inflated to a working configuration.

One of the main functions of balloons is to open up a constriction. Such a constriction may be caused by remodelling of tissues, such as remodelling of vessel walls, for example as in stenosis (including restenosis), or blockage such as by material build-up such as build-up of plaque and/or vessel wall thickening such as might occur with for example atherosclerosis. The constriction may also be in a valve, for example a heart valve. A balloon may be used in a procedure, such as a valvuloplasty procedure, to unblock a valve, and/or to open it up, for example by stretching the valve tissue, for example stretching the valve annulus. In some cases the valve may have become calcified and stiff thus restricting its opening. A balloon is used to stretch the valve.

For example angioplasty balloons are used to open up diseased arteries and thus restore blood flow.

A second main function of balloons is for delivery. For example a balloon may be used to deliver a collapsed device which is for retention within the body, and then to expand the device from its collapsed position at a target site so that it is deployed for use. For example, it is known to use balloons to deploy stents at target sites. It is also known to use balloons to deploy valves. It is also known to use balloons to deploy filters.

Many types of balloons have been developed for medical interventions. These include compliant balloons and non-compliant balloons. Typically non-compliant materials are utilised to form balloons that expand by unfolding. Such balloons are often typically inflated to higher pressures. Such balloons have a predetermined diameter which is defined by the non-compliant material when it is unfolded and inflated. Furthermore, these balloons tend to inflate to a predetermined shape.

Balloons formed of compliant materials can be inflated to different diameters using different pressures. Often times they can expand to several times their unexpanded size. Because they are compliant they do not tend to maintain a regular shape when being inflated.

Some balloons have been provided with sheaths from which they are unsheathed in order to be deployed. Most are associated with a catheter that introduces the balloon. A catheter will often have a lumen defined therein which is utilised for inflation.

A balloon with multiple diameters is produced by Boston Scientific and is sold under the trade mark CRETM (Controlled Radial Expansion Dilator). CRETM has three distinct, pressure controlled diameters (6-20 mm, 3-6 ATM) and is labelled for use in oesophageal (throat), pyloric (small intestine) and colonic (large intestine) applications. This device is outlined and protected under US2001/0008970, which is a continuation of US1995/5766201 filed on 7th Jun 1995. A strip is wrapped in a helical fashion about the balloon such that the strip is stretched when the balloon is expanded to its expanded profile. This device is not suitable for angioplasty procedures due to its large diameter and may not have the level of control required for such procedures.

Reinforced balloons are also known for example from US2008/0255512; US2009/0043254 and US2009/00038752.

A catheter balloon, designed to be expanded to two different, known work-hardened diameters, is described in US1995/5681343. Methods and systems have been developed to adjust the length of a catheter balloon, are described in US1994/5549551 and US2003/6527741.

US1996/5843027, US1996/6090072 and US2001/0008970 describe a tubular sheath or sleeve that fits over the balloon thereby providing greater control over expansion and inflation.

Asymmetrical balloon designs have been disclosed in US1994/5470313 which combines differing material properties (elastic modulus, compliance and deformation force) into one balloon. The balloon combines a ridged high-pressure type balloon configuration, which remains within its elastic limit, with a low-pressure balloon type configuration, wherein a section of the balloon is stretched into plastic deformation.

US2000/6488653 and US2004/0064064, describe balloons with multiple diameters along their lengths. These devices have varying lengths associated with each of their diameters and expansion of all diameters occurs simultaneously.

Multiple balloons, aligned in different configurations, so as to give combined inflation diameters or single balloons containing multiple chambers which are independently inflatable have been disclosed in US1986/4744366; US1986/4763654, US2004/0209674; US1992/5304135 and US2007/0213663. The cost associated with the manufacture of these devices, however, may be prohibitive for their commercial implementation.

US2003/0075711 describes a device designed with multiple layers of balloons.

Notwithstanding the choices available it is desirable to provide an alternative construction.

### SUMMARY OF THE INVENTION

The present invention provides an inflation balloon comprising:
(i) a compliant balloon wall;
(ii) a first set of non-compliant filamentous members within the balloon wall for restraining the balloon to a first expanded size,
(iii) a second set of non-compliant filamentous members within the balloon wall for restraining the balloon to a second larger expanded size,
   wherein the first set of non-compliant filamentous members are arranged to undergo failure to allow the balloon to expand from the first expanded size to the second larger expanded size.

This forms a balloon that is suitable for use in a medical procedure. For example the balloon of the invention can be introduced into the body via a catheter.

The characteristics of such a balloon are a hybrid of those of a compliant and non-compliant balloon. Unlike a compliant balloon, the balloon of the invention has set sizes (diameters). In particular, it can be inflated until it is restrained by the first set of non-compliant filamentous members. Once in this configuration where it is restrained, it behaves like a non-compliant balloon.

Once a threshold pressure is exceeded, the first set of non-compliant filamentous members fail. This allows the balloon to expand until it is restrained by the second set of non-compliant filamentous members. Such an expansion would not typically be achievable with a single non-compliant balloon. So between the first and second expanded sizes, the balloon once again behaves like a compliant balloon—it can expand.

Its characteristics are comparable to a non-compliant balloon having two different operational sizes.

This is achieved with a single balloon. In the past multiple balloons would have to be used in order to achieve different sizes.

The balloon can be operated at two different sizes, for example used in a medical procedure at the first (smaller) size and then later at the second (larger) size. In this way, sequential and increasing expansive force can be utilised. However, unlike a non-compliant balloon, the size of the balloon is predetermined based on the first and second set of non-compliant members. The first and second set of non-compliant members are selected to impart respective desired sizes to the balloon. This means that the balloon can be manufactured to inflate to first and second desired sizes based on the end-use application. It is clear also that use of the different sizes can impart different degrees of force to a target site.

For any operational size of a balloon of the invention, the first and second (and indeed any subsequent) sizes imparted by first or second (or subsequent) sets of non-compliant members will all be within a safe inflation (size and pressure) range for the balloon. This ensures that there is no danger of rupture of the balloon itself at any of its operational sizes.

The present invention provides an inflatable balloon (suitable for use within the body for example within the vasculature) comprising:
a compliant balloon wall having an inflation size limit beyond which it ruptures; a set of filamentous members arranged within the balloon wall so as to restrict the balloon to a first balloon inflation size which is less than the inflation size limit; and to fail at a predetermined inflation pressure so as to no longer restrict the balloon to a first balloon inflation size thus allowing the balloon to be inflated to a second size that is greater than the first inflation size the second size being no greater than the inflation size limit.

The present invention provides an inflation balloon comprising:
(i) a compliant balloon wall;
(ii) a first set of non-compliant filamentous members within the balloon wall, the filamentous members being arranged to be slack so as to allow inflation of the inflation balloon, but to restrain the balloon to an expanded diameter when all of the slack has been taken up by expansion of the balloon;
(iii) a second set of non-compliant filamentous members within the balloon wall, the filamentous members being arranged to be slack so as to allow inflation of the inflation balloon wherein the first non-compliant filamentous members are adapted to restrain the balloon to a first expanded diameter and the second non-compliant filamentous members are adapted to restrain the balloon to a second expanded diameter.

The present invention provides an inflation balloon comprising:
(i) a compliant balloon wall;
(ii) a set of non-compliant filamentous members within the balloon wall, the filamentous members being arranged to be slack so as to allow inflation of the inflation balloon, but to restrain the balloon to an expanded diameter when all of the slack has been taken up by expansion of the balloon.

A given set of non-compliant filamentous members will comprise at least two, for example, two or three, such as at least three non-compliant filamentous members.

Desirably there are a plurality of sets of non-compliant filamentous members. There will be at least two sets of non-compliant filamentous members and optionally at least three.

Desirably the non-compliant filamentous members are embedded within the balloon wall.

Each filamentous member may be in the form of a ring. Desirably, the sets of non-compliant filamentous members are formed by sets of (independent) rings. These may be spaced apart from each other. However, any given set of non-compliant filamentous members will be designed to act in the same manner, in particular, to provide a uniform dimension/size to at least a selected part of the balloon.

It will be appreciated then that the present invention provides an inflation balloon which has a size which can be stepped. The present invention allows for multistage inflation.

The construction of the present invention provides good control over the inflation size, for example diameter of an inflation balloon. Such inflatable balloons are suitable for use in balloon catheter devices.

The non-compliant filamentous members impart control of the balloon within a normal expansion range of the balloon and in particular within a range that does not include failure of the balloon.

While the prior art designs give some control over the inflation size, for example diameter of a balloon catheter device in angioplasty procedures, many procedures require multiple inflations and deflations such that the clinician has precise control over the size, for example diameter. This requires multiple sizes/diameters to be achieved uniformly along the length of the balloon throughout the procedure. The prior art does not achieve this. However, this may be achieved with the constructions of the present invention.

Also, the advantages of the present invention are achieved with a single balloon construction. In particular, there is no requirement to have multiple balloons of different sizes. In particular, a balloon of the invention can be constructed of a compliant material, yet still be provided with different areas/portions with different diameters if required. Also, a balloon of the invention can be restrained to a first (smaller) size, for example diameter, and then, the first (set of) non-compliant filamentous members may be subjected to a pressure which causes them to fail. Once they fail, the balloon can expand again. This provides at least two different operating diameters/sizes for the balloon of the invention. Failure of the first (set of) non-compliant filamentous members does not cause failure of the balloon. It is still operational.

A second set of non-compliant filamentous members can be utilised then to restrain the balloon of the invention to a second (greater) diameter. Failure of the second set of non-compliant filamentous members does not cause failure of the balloon.

One simple way of achieving the effect of the present invention, is to provide the first (set of) non-compliant filamentous members in an arrangement where they follow a non-linear path around, for example circumferentially around, the balloon wall (in a deflated state of the balloon). In this way they do not immediately experience any substantial load as the balloon starts to inflate. Furthermore they do not interfere to any substantial extent with inflation of the balloon. However, when the balloon reaches the first size/diameter, the (first set of) non-compliant filamentous members then become taut. They then restrain the balloon to the size/diameter.

The filamentous members may be arranged so that as the balloon is inflated the filamentous members move toward a linear path, without interfering with the inflation of the balloon, and when their maximum extension is reached they restrain the balloon against further inflation.

For example, the filamentous members may be arranged to follow a wave-like path or pattern. As the balloon expands the wave-like form is pulled taut and tends to flatten out (reducing the amplitude of the wave pattern and tending towards a straight line). Once pulled tight by the expansion of the balloon the filamentous members restrict further expansion of the balloon.

As mentioned above, within an inflation balloon of the invention there may be a first set of non-compliant filamentous members and a second set of non-compliant filamentous members each being arranged to be slack so as to allow inflation of the inflation balloon wherein the first set of non-compliant filamentous members are adapted to restrain the balloon to a first expanded size, for example diameter and the second set of non-compliant filamentous members are adapted to restrain the balloon to a second expanded size, for example diameter.

It will be appreciated that if a second set of non-compliant filamentous members are designed to fail, then they fail at a pressure threshold which is greater than the pressure threshold at which the first set of non-compliant filamentous members fail.

This means that different sets of non-compliant filamentous members may be utilised to restrain the balloon to a different size, in different parts of the same balloon, for example a balloon which has a portion with a size, for example diameter larger than another. In such an arrangement the first set of non-compliant filamentous members and the second set of non-compliant filamentous members are arranged in different parts of the balloon so that the balloon when inflated has different parts with different sizes/diameters. Indeed, it is envisaged that many different shapes could be imparted to the balloon by utilising two, three, or even more different sets of non-compliant filamentous members.

The first set of non-compliant filamentous members may be arranged to undergo failure before the balloon can expand from the first expanded size, for example diameter to the second expanded size, for example diameter. The construction of the invention allows (consecutive and/or stepwise) failure of first, second, and if desired, later generations of non-compliant filamentous members.

This means that a single balloon can have multiple working dimensions. The transition from an earlier generation of non-compliant filamentous members to a later generation occurs when the set of non-compliant filamentous members in an earlier generation fails. A given set of non-compliant filamentous members can be designed to fail at a predetermined (e.g. tensile pressure) threshold. So for example, an inflation balloon can be designed for use in a medical intervention where it has two or more inflation stages. This obviates the necessity for utilising different balloons of different sizes. For example a balloon can be inflated to a first dimension to stretch tissue, for example open a constriction in a vessel. Then additional pressure is applied and the first set of non-compliant filamentous members fail. The balloon then inflates to a second size for example one where the size is determined by a second set of non-compliant filamentous members. This means the same balloon can be used to stretch the same tissue even further. This obviates the necessity to use two different devices, one with a first (smaller) balloon and then a second with a larger balloon.

Any failure that occurs of non-compliant filamentous members is desirably brittle failure. This means that there is relatively little deformation of the non-compliant filamentous members before they fail. This in turn allows good control of the size of the balloon during inflation, and during failure.

For example it is desirable that the non-compliant filamentous members deform by less than 10% when under a tensile load. Desirably they fail under tensile load and optionally by fracturing across their cross-section.

It will be appreciated that there are many ways of programming different fail points into an inflation balloon of the invention.

For example different materials having different fail points (e.g. tensile strengths) can be used. Different thicknesses of materials can be used. Different lengths can be used. And combinations of same are also possible. The present invention provides an inflation balloon of the invention wherein the first set of non-compliant filamentous members and the second set of non-compliant filamentous members differ from each other by being of different lengths. They will also differ from each other by failing at different (threshold) pressures.

It will be appreciated that depending on the arrangement different lengths (of otherwise similar non-compliant filamentous members) can be used to programmed different characteristics into the balloon.

For example utilising an arrangement wherein the first set of non-compliant filamentous members and the second set of non-compliant filamentous members differ from each other by being of different lengths but which fail at the same tensile force, one can provide them in an arrangement, for example where they are provided in different parts of the inflation balloon, where they allow the balloon to be expanded so that one part of the balloon has different dimensions imparted to it than to another part. The sets of non-compliant filamentous members operate in series so that one part of the balloon has a greater size, for example diameter to another, yet both sets of filamentous members undergoes (brittle) failure at the same tensile force. For example a shaped balloon can have a first set of non-compliant filamentous members and a second set of non-compliant filamentous members which impart a different size, for example diameter to the balloon but both of which fail at the same applied (tensile) force. The balloon can then be allowed to expand to dimensions imparted by one or more further sets of non-compliant filamentous members. These further non-compliant filamentous members could include two sets of non-compliant filamentous members which also impart different size, for example diameters. Or these further non-compliant filamentous members could include one set that imparts the same size, for example diameter so that the shape (difference) imparted by the first and second set of non-compliant filamentous members is lost (or programmed out).

Utilising an arrangement wherein the first set of non-compliant filamentous members and the second set of non-compliant filamentous members differ from each other by being of different lengths and/or diameter and by failing at a different tensile force the filamentous members can fail in sequence, with failure of the first set of non-compliant filamentous members required before the balloon can expand to a size determined by the second set of non-compliant filamentous members. Failure can thus occur across an ordered set of inflation pressures/tensile strengths, for example two or more distinct inflation pressures/tensile strengths.

It will be appreciated that these approaches can be combined.

For example a balloon having two different sizes/diameters may only undergo failure in one part. For example a balloon with a larger size, for example diameter and a smaller size, for example diameter can have failure of a set of non-compliant filamentous members in the part with the smaller size, for example diameter allowing that part to expand to the same dimensions as the larger size, for example diameter part. (This could be achieved for example by having a first set of non-compliant filamentous members in only a part of the balloon, while a second set of non-compliant filamentous members are provided throughout the entire balloon.) A balloon with two different sizes, for example diameters may only undergo failure in one part. For example a balloon with a larger size, for example diameter and a smaller size, for example diameter can have failure of the set of non-compliant filamentous members in the part with the larger size, for example diameter allowing that part to expand to have even greater dimensions relative to the smaller size part. A balloon having the same size, for example diameter can undergo failure to create a balloon with a larger size, for example diameter in a part or in the whole of the balloon.

The basic principle then is that different sets of non-compliant filamentous members can be used to impart different sizes/diameters to different parts of a balloon. Failure of different sets of non-compliant filamentous members can also be used to impart different sizes/diameters to a balloon. Failure can also be used to remove differences in size, for example diameter in a balloon.

The first set of non-compliant filamentous members may be in the form of rings that run about a circumference of the balloon.

The second set of non-compliant filamentous members may be in the form of rings that run about a circumference of the balloon.

The filamentous members upon maximum extension restrain the balloon in first dimensions suitable for operation thereof.

Having balloons with different sizes/diameters can be useful where there are bifurcations in the target site for example bifurcated vessels within the vasculature.

Any given set of non-compliant filamentous members do not interfere to any great extent with inflation of the balloon. They only restrict further expansion when they become tensioned. They only become tensioned when all of the slack is taken up. Before they restrict expansion they are in a substantially untensioned or relaxed state. For example they may only reach their tensioned state under a given inflation pressure.

The filamentous members are desirably monofilament materials such as monofilament fibres.

The invention also provides a medical device for insertion into the human or animal body comprising an inflation balloon of the invention.

The invention also relates to a method of operation of a device of the invention comprising, providing a device of the invention, inserting it into the human or animal body, inflating the inflation balloon for example to impart a stretching force.

Desirably the balloon is constructed from the following materials: latex or polyisoprene, silicone, polyurethanes and combinations thereof. The latex may be a natural or synthetic material or a combination of natural and synthetic materials.

Desirably the balloon wall is between 20 and 1000 μm thick.

Desirably the balloon has a first diameter which is about 2 to about 40 mm in length. Desirably it expands to a second diameter which is about 2 to about 40 mm in diameter.

Desirably the non-compliant filamentous members may be constructed from the following materials: silicon; nylon, silicon-PTFE tempered monofilament (and alternatively tempered monofilaments); PTFE (polytetrafluoroethylene); PEBA (polyether block amide) for example material sold under the trade name PEBAX; PET (polyethylene terephthalate); polyurethanes and combinations thereof. Metals (including alloys) may also be considered, such as nitinol filaments.

Desirably the non-compliant filamentous members may be from about 6 to about 250 mm in length.

Desirably the non-compliant filamentous members may be from about 0.01 to about 1 mm in thickness.

Desirably the non-compliant filamentous members may be selected to fail at a tensile pressure from about 200 to about 2634 kPa (2 to about 26 atmospheres).

### BRIEF DESCRIPTION OF THE DRAWINGS

Embodiments of the invention will be described, by way of example only, with reference to the accompanying drawings in which:

FIG. 1 is a schematic representation of a conventional inflation balloon;

FIG. 2A is a schematic representation of an inflation balloon of the invention with a set of non-compliant filamentous members embedded in the balloon wall;

FIG. 2B is a schematic representation of an inflation balloon of FIG. 2A after inflation and with the set of non-compliant filamentous members under tension restricting the inflation of the balloon;

FIG. 3A is a schematic representation of an inflation balloon of the invention with two sets of non-compliant filamentous members embedded in the balloon wall;

FIG. 3B is a schematic representation of an inflation balloon of FIG. 3A after inflation and with a first set of non-compliant filamentous members under tension restricting the inflation of the balloon, but with a second set of non-compliant filamentous members which are not under tension and are not restricting the inflation of the balloon;

FIG. 3C is a schematic representation of an inflation balloon of FIG. 3A after inflation and with a first set of non-compliant filamentous members having failed, and with the second set of non-compliant filamentous members under tension restricting the inflation of the balloon;

FIG. 4A is a schematic representation of an inflation balloon of the invention with two sets of non-compliant filamentous members embedded in the balloon wall each of the sets of non-compliant filamentous members being different parts of the balloon;

FIG. 4B is a schematic representation of the inflation balloon of FIG. 4A with both sets of non-compliant filamentous members under tension restricting the inflation of the balloon and conferring a stepped size, for example diameter on the balloon;

FIG. 5A is an image of a latex test sample in a “dog bone” shape;

FIG. 5B is an image of a latex test sample in a “dog bone” shape with an embedded non-compliant filamentous member in the form of a fibre of monofilament silicon and with arrows indicating the pattern of the non-compliant filamentous members/fibre pattern;

FIG. 6A is an image of the crimping tool used in the experimental part below;

FIG. 6B is a schematic representation of the crimping tool of FIG. 6A giving lengths in millimetres;

FIG. 7 Stress (MPa) Vs Elongation (%) of latex (dog-bone shaped) samples;

FIG. 8 is a plot of Stress (MPa) Vs Elongation (%) of uncrimped 0.08 mm silicon non-compliant filamentous members/fibres;

FIG. 9 is a plot of Stress (MPa) Vs Elongation (%) of latex samples with embedded 0.08 mm diameter silicon non-compliant filamentous members/fibres;

FIG. 10 is a plot of Stress (MPa) Vs Elongation (%) of latex with embedded 0.08 mm diameter silicon non-compliant filamentous members/fibres;

FIG. 11 is a plot of Stress (MPa) Vs Elongation (%) of latex with two sets of embedded 0.08 mm diameter silicon non-compliant filamentous members/fibres;

FIG. 12 is a plot of Stress (MPa) Vs Elongation (%) of latex with two sets of embedded 0.08 mm diameter silicon non-compliant filamentous members/fibres;

FIG. 13 is a scanning electron microscope (SEM) cross section view of a latex sample. No defects between the boundary layers can be seen across multiple samples. The lines highlight three layers of the sample; and

FIG. 14(a) is an image showing a cylindrical balloon formed and inflated to a diameter of 5 mm approximately (as indicated by the callipers) and FIG. 14(b) shows the same balloon inflated to a diameter of approximately 8 mm.

### DETAILED DESCRIPTION OF THE DRAWINGS

In this study, novel materials for use in multiple size/diameter balloons for use in biomedical applications are constructed. Such balloons have advantages over current technology in specific applications such as angioplasty/valvuloplasty procedures at bifurcations, in vessels with significantly varying diameters or to deliver, deploy and position transcatheter heart valves.

These novel balloons utilise monofilament fibres or non-compliant filamentous members to control the expansion of the balloon and allow multiple set expansion sizes, for example diameters in one balloon. This study shows that by adjusting the undulation pattern of the monofilament non-compliant filamentous members/fibres, the expansion, shape and number of sizes/diameters can be controlled.

Angioplasty balloons are used to open up diseased arteries and restore blood flow. In many cases a small size, for example diameter angioplasty balloon is required for the initial expansion of the arterial lumen followed by one or more, larger size, for example diameter angioplasty balloons to completely restore blood flow in the artery.

Balloon heart valve surgery (valvuloplasty) is a procedure for opening a blocked heart valve. This procedure uses a balloon to stretch the valve or to break up scars in the valve. It may be done in conjunction with valve replacement, instead of conventional open surgery, whereby a Transcatheter Aortic Valve Replacement (TAVR) procedure is carried out.

TAVR is a procedure for select patients with severe symptomatic aortic stenosis. TAVR is performed on a beating heart and does not require cardio-pulmonary bypass. A catheter is placed in the femoral artery (in the groin) similar to angioplasty, and guided into the chambers of the heart. A crimped (compressed) tissue heart valve is placed on the balloon catheter and is positioned directly inside the diseased aortic valve. Once in position, the balloon is inflated to secure the valve in place. This procedure can require the use of multiple balloons to ensure the valve is held securely in place during delivery, adequately positioned and then subsequently fully deployed.

The use of a multiple size/diameter/shape balloon to deploy stents and valves would reduce procedural time and allow more effectively delivery these devices.

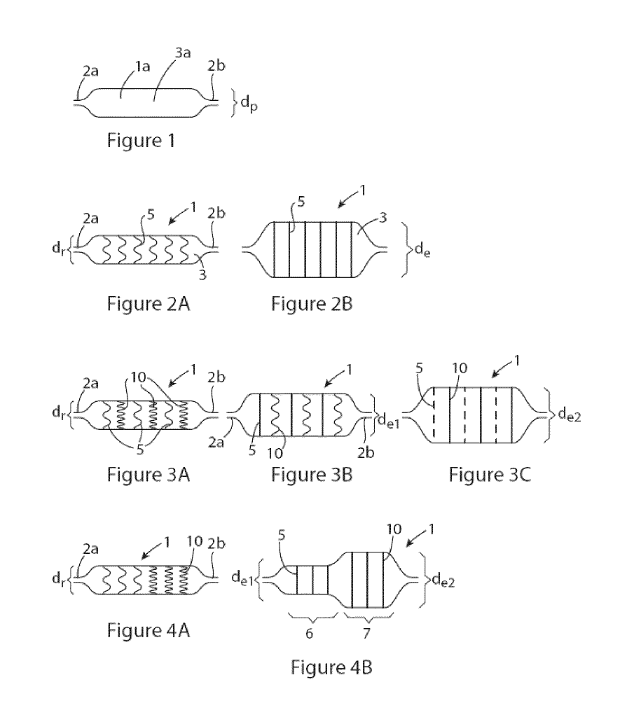

FIG. 1 shows a schematic representation of a conventional inflation balloon 1a. It has a compliant balloon body or wall 3a. It has been inflated to a diameter dₚ and it has ends 2a and 2b where it is attachable to a catheter.

FIG. 2A is a schematic representation of an inflation balloon 1 of the invention, in an uninflated condition. It has a compliant balloon body or wall 3. It has ends 2a and 2b where it is attachable to a catheter. (It will be appreciated that the balloon 1 is substantially cylindrical in cross-sectional shape.)

Embedded in the balloon wall 3 and in particular embedded within the balloon wall 3 are a set of non-compliant filamentous members 5. The non-compliant filamentous members 5 are shown in a slack or relaxed or untensioned configuration. The non-compliant filamentous members 5 follow a non-linear path along the balloon wall 3 (in a deflated state of the balloon 1). In this state the balloon 1 has a diameter dᵣ. In this way the non-compliant filamentous members 5 do not immediately experience any substantial load as the balloon 1 starts to inflate. Furthermore they do not interfere to any substantial extent with inflation of the balloon 1. However, when the balloon 1 reaches a first size/diameter dₑ as shown in FIG. 2B, the non-compliant filamentous members 5 then become taut. They then restrain the balloon 1 to the size/diameter dₑ as shown in FIG. 2B. The diameter dₑ is greater than the diameter dᵣ.

The non-compliant filamentous members 5 are arranged so that as the balloon 1 is inflated the filamentous members move toward a linear path, without interfering with the inflation of the balloon, and when their maximum extension is reached they restrain the balloon 1 against further inflation.

The non-compliant filamentous members 5 follow a wave-like path or pattern as shown in FIG. 2A. As the balloon 1 expands the wave-like form is pulled taut and tends to flatten out (tending towards a straight line). Once pulled tight by the expansion of the balloon 1 the filamentous members 5 restrict further expansion of the balloon.

FIG. 3A is a schematic representation of an inflation balloon 1 of the invention with two sets of non-compliant filamentous members 5, 10 embedded in the balloon wall 3. The non-compliant filamentous members 5, 10 are arranged in sequence (are nested) so that the filamentous members 5 must fail before the balloon 1 can expand to a dimension imparted by the non-compliant filamentous members 10. In FIG. 3A the balloon has a diameter dᵣ. As inflation pressure increases the balloon 1 initially expands to the configuration shown in FIG. 3B where it has a first diameter dei imparted by the first set of non-compliant filamentous members 5. In this configuration the second set of non-compliant filamentous members 10 are not imparting any shape restriction on the balloon 3. FIG. 3C is a schematic representation of an inflation balloon 1 of FIG. 3A after inflation and with the first set of non-compliant filamentous members 5 having failed (failure is indicated by the broken lines shown), and with the second set of non-compliant filamentous members 10 under tension restricting the inflation of the balloon. In the configuration of FIG. 3C the balloon 1 has a diameter dₑ₂. The diameter dₑ₂ is greater than the diameter dₑ₁ which is greater than the diameter dᵣ. The balloon of FIGS. 3A to 3C can thus be inflated in a multi-staged arrangement. The diameter can be stepped up from one configuration to another.

FIG. 4A is a schematic representation of an inflation balloon 1 of the invention with two sets of non-compliant filamentous members 5, 10 embedded in the balloon wall 3. Again the balloon has an uninflated diameter dᵣ. Each of the sets of non-compliant filamentous members 5, 10 are in different parts of the balloon 1. First set of non-compliant filamentous members 5 and the second set of non-compliant filamentous members 10 are arranged in different parts of the balloon 1 so that the balloon when inflated has different parts with different sizes/diameters.

FIG. 4B is a schematic representation of the inflation balloon 1 of FIG. 4A with both sets of non-compliant filamentous members 5, 10 under tension restricting the inflation of the balloon and conferring a stepped size/diameter on the balloon 1. In particular the non-compliant filamentous members 5 restrict the part 6 of the balloon 1 to a diameter dₑ₁ and the non-compliant filamentous members 10 restrict the part 7 of the balloon 1 to a diameter dₑ₂.

It will be appreciated that the configurations illustrated can be combined in any desired way as described above.

The non-compliant filamentous members are in the form of rings that run about a circumference of the balloon 1.

### Experimental

Test Sample Composition

Two test sample compositions were created, samples B001 and B002. B001 is a traditional latex compliant balloon material with no additives. B002 is a latex compliant balloon material which contains 0.08 mm monofilament Silicon.

B001 samples were created by setting latex (Acrylic latex) in 0.1 mm layers and allowing the Latex to cure at an ambient temperature for 12 hours, before additional layers were added using the same process to have a finished sample of thickness 0.8 mm. B002 samples were created using the same latex used in B001 samples. A first layer was poured to a depth of 0.1 mm and allowed to cure at an ambient temperature for 12 hours. A second, third and fourth layer were added using this method. A crimped non-compliant filamentous member in the form of a silicon fibre (0.08 mm diameter, Silicon-PTFE tempered Monofilament) was embedded into the fourth layer before the fifth layer was poured and allowed to cure. Three addition layers were poured in the same process to complete the samples and give them a thickness of 0.8 mm.

Latex Testing

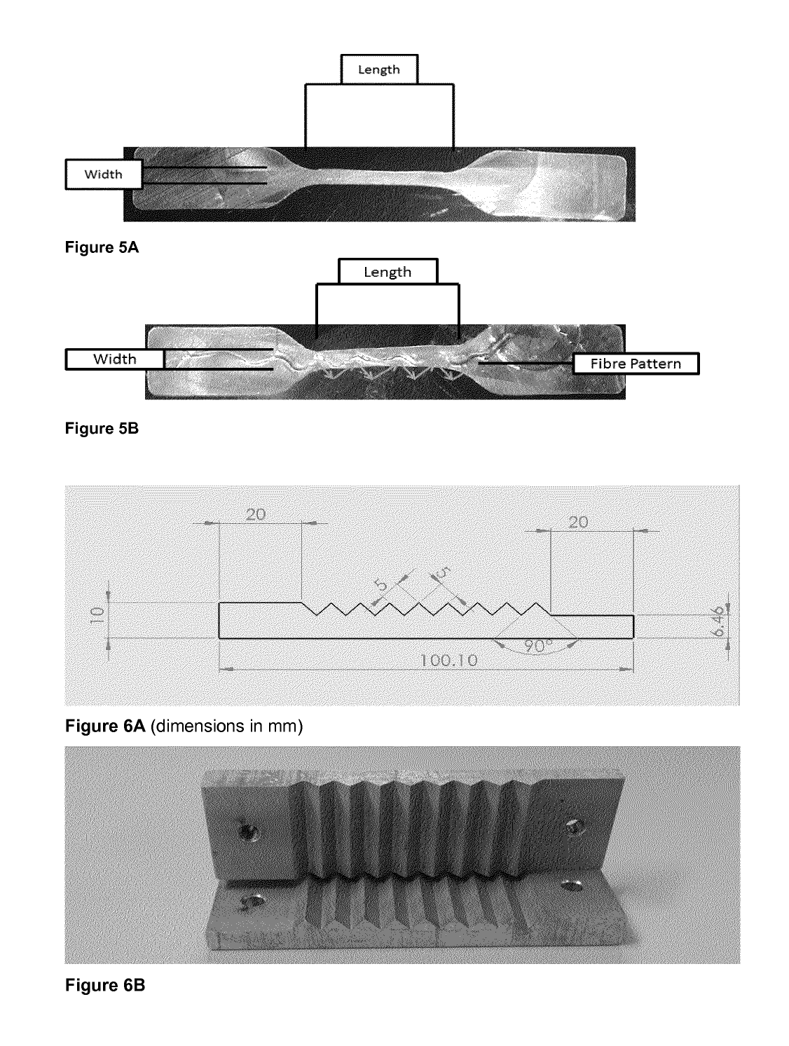

To test the Latex in use, each material was formed into dog-bone shaped samples. Sample B001 is shown in FIG. 5A. Sample B002 is shown in FIG. 5B. Uniaxial tensile testing was conducted on the test specimens. A Zwick 5kN Z005 machine was used to test all samples. The tensile tests were based on ISO 15037 and ASTM D828.

The samples had a length 27.6 mm (this is the length indicated in FIGS. 5a and 5B); a width of 2.4 mm (this is the width indicated in FIGS. 5a and 5B) and a thickness of 0.8 mm.

The results of the tests are broken down below to show key points upon expansion.

Monofilament Testing

To test the monofilament silicon, silicon monofilament (0.08 mm diameter, Silicon-PTFE tempered Monofilament),was cut to lengths of 100 mm and crimped using a ninety degree crimping tool, which is shown in FIG. 6A and the dimensions for which are given in FIG. 6B. Uniaxial tensile testing was conducted on the test specimens. The Zwick Z005 was used to test all samples.

Scanning electron microscope (SEM) analysis of latex layers.

SEM analysis was carried out using a Hitachi S-3400N. Latex samples were sectioned from the latex test specimens and fixed to viewing plates. To allow a clear picture to be seen, the samples were placed on a gold leaf base to conduct electrons away from the surface and prevent charge build-up on the sample.

Results

Latex Testing

TABLE 1
B001, Tensile test data.

| | Ultimate Tensile Strength | Elongation at UTS |
|---|---|---|
| Sample | (UTS) (MPa) | (%) |
| 1 | 1.13 | 965 |
| 2 | 0.73 | 652 |
| 3 | 1.37 | 788 |
| 4 | 0.85 | 755 |
| 5 | 1.27 | 968 |
| 6 | 1.41 | 972 |
| Range | 0.73-1.41 | 652-972 |

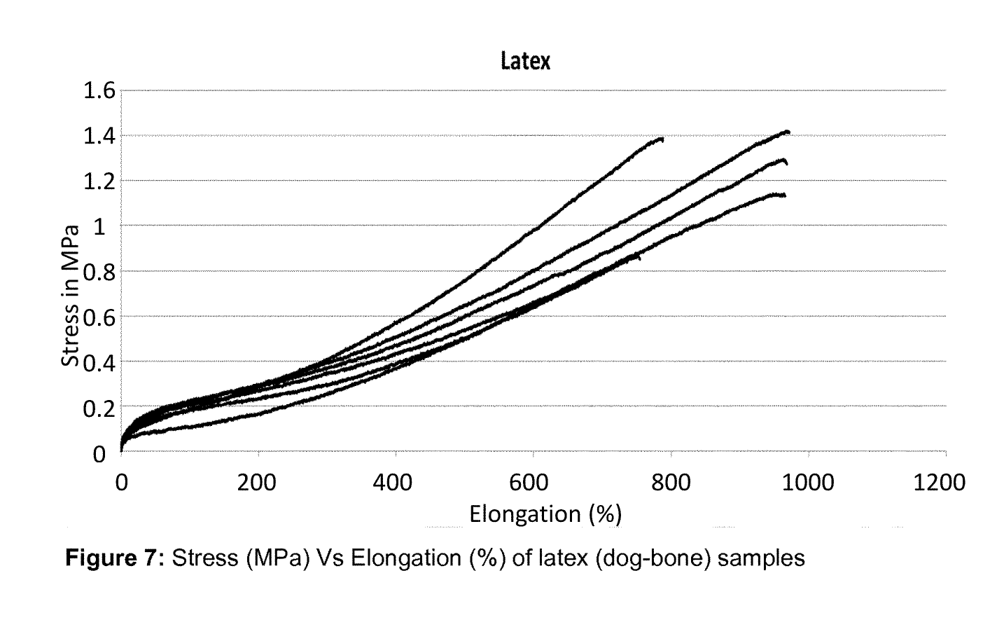

FIG. 7 is a plot of Stress (MPa) Vs Elongation (%) for the latex (dog-bone shaped) samples.

Monofilament Testing

TABLE 2
0.08 mm crimped silicon fibres, tensile test data.

| | Maximum Engineering Stress | Elongation |
|---|---|---|
| Sample | (MPa) | % |
| 1 | 3.94 | 8 |
| 2 | 3.45 | 6 |
| 3 | 3.54 | 7 |
| 4 | 3.49 | 7 |
| 5 | 3.58 | 8 |
| 6 | 2.62 | 8 |
| Range | 2.62-3.94 | 6-8 |

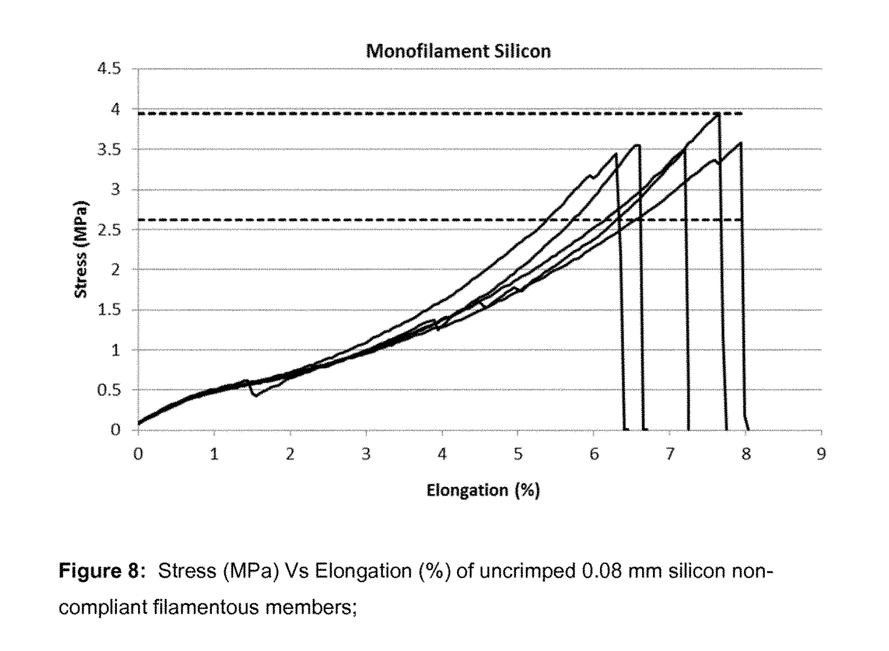

FIG. 8 is a plot of Stress (MPa) Vs Elongation (%) of uncrimped 0.08 mm silicon fibres

TABLE 3
B002, Tensile test data.

| | Engineering Stress (MPa) at | Elongation (%) at |
|---|---|---|
| | failure of latex with embedded | failure of latex with embedded |
| | set of non-compliant | set of non-compliant |
| Sample | filamentous members | filamentous members |
| 1 | 0.86 | 50 |
| 2 | 0.68 | 44 |
| 3 | 0.81 | 48 |
| 4 | 0.61 | 52 |
| 5 | 0.80 | 36 |
| 6 | 0.72 | 23 |
| Range | 0.61-0.86 | 23-52 |

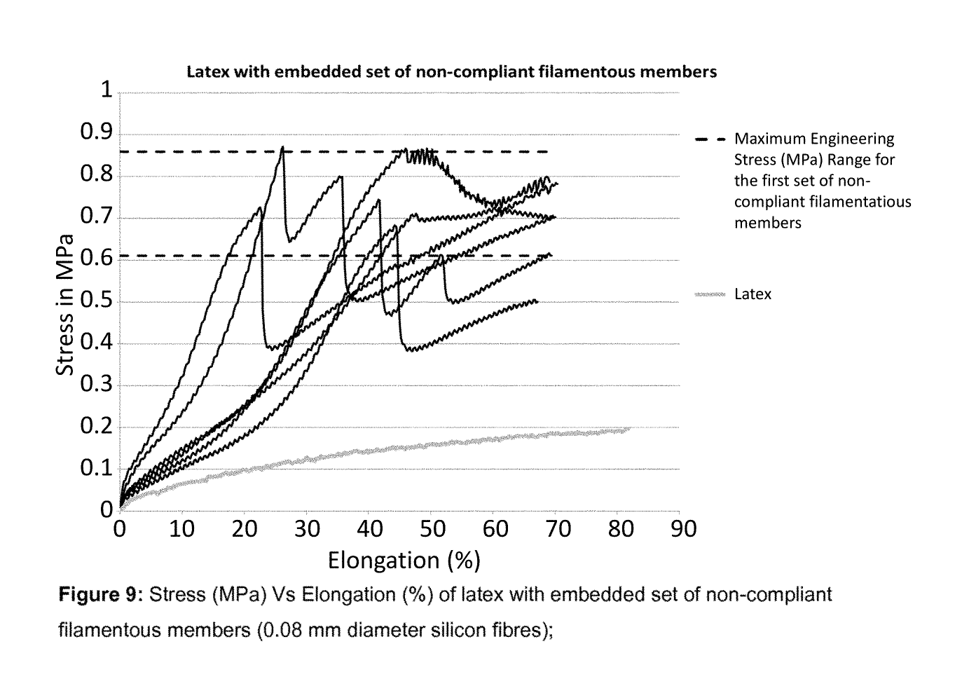

FIG. 9 is a plot of Stress (MPa) Vs Elongation (%) of latex samples with embedded 0.08 mm diameter silicon non-compliant filamentous members/fibres.

FIG. 9 shows latex samples with embedded non-compliant filamentous members/fibres undergoing uniaxial tensile testing. Each sample demonstrates the failure of the embedded non-compliant filamentous members/fibres and the subsequent strain hardening of the latex/non-compliant filamentous members/fibres combination material following. The maximum engineering stress (MPa) range is highlighted by dashed lines. A comparison to natural latex is shown at the bottom of the graph to highlight the difference between the new test sample and a latex sample. It is worth noting that failure of the non-compliant filamentous members/fibres does not result in failure of the sample overall.

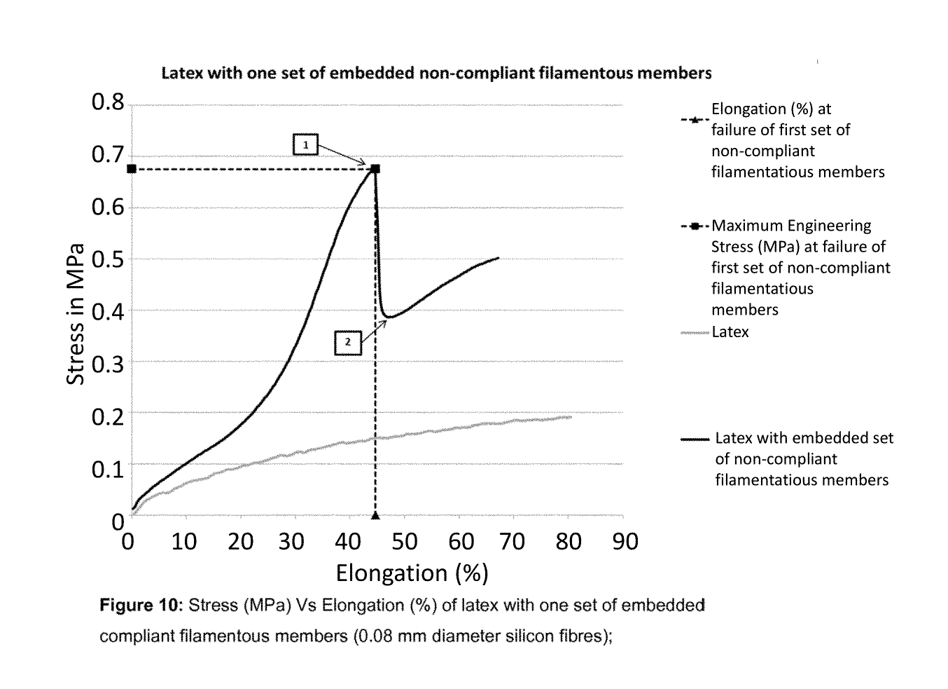

Arrow marker 1 in FIG. 10 shows the stress (MPa) at failure of the embedded non-compliant filamentous member/fibre. The embedded non-compliant filamentous member/fibre controls the elongation (%) of the sample with respect to stress (MPa) until the non-compliant filamentous member/fibre fails; at this point a large drop in stress acting on the sample is seen. This drop in stress (MPa) is because the non-compliant filamentous member/fibre is no longer intact to resist the uniaxial elongation (%) of the sample and therefore less stress (MPa) is required to elongate the sample. This causes strain hardening in the material and this is shown by arrow marker 2.

The key take away from FIG. 10, is the controlled expansion of the sample up to the failure point of the non-compliant filamentous member. Shown in FIG. 9, the sets of non-compliant filamentous members all fail within a small stress (MPa) range between 0.61-0.86 (MPa). Based on this, elongation of the sample can be controlled by the crimping pattern of the non-compliant filamentous members/fibres up to the point of failure. The non-compliant filamentous members/fibres extend to their full length and exhibit a brittle failure on full expansion.

TABLE 4
B003, Tensile test data.

| | Engineering | Engineering | Elongation | Elongation |
|---|---|---|---|---|
| | Stress (MPa) | Stress (MPa) | (%) at | (%) at at |
| | at failure | at failure | failure | failure |
| | of first | of second | of first | of second |
| | set of non- | set of non- | set of non- | set of non- |
| | compliant | compliant | compliant | compliant |
| | filamentous | filamentous | filamentous | filamentous |
| Sample | members | members | members | members |
| 1 | 1.04 | 1.08 | 68 | 84 |
| 2 | 1.18 | 0.92 | 79 | 100 |
| 3 | 1.25 | 1.13 | 76 | 97 |
| 4 | 1.13 | 0.92 | 70 | 101 |
| 5 | 1.19 | 0.93 | 67 | 103 |
| 6 | 1.01 | 0.92 | 70 | 85 |
| Range | 1.01-1.25 | 0.92-1.13 | 67-79 | 84-103 |

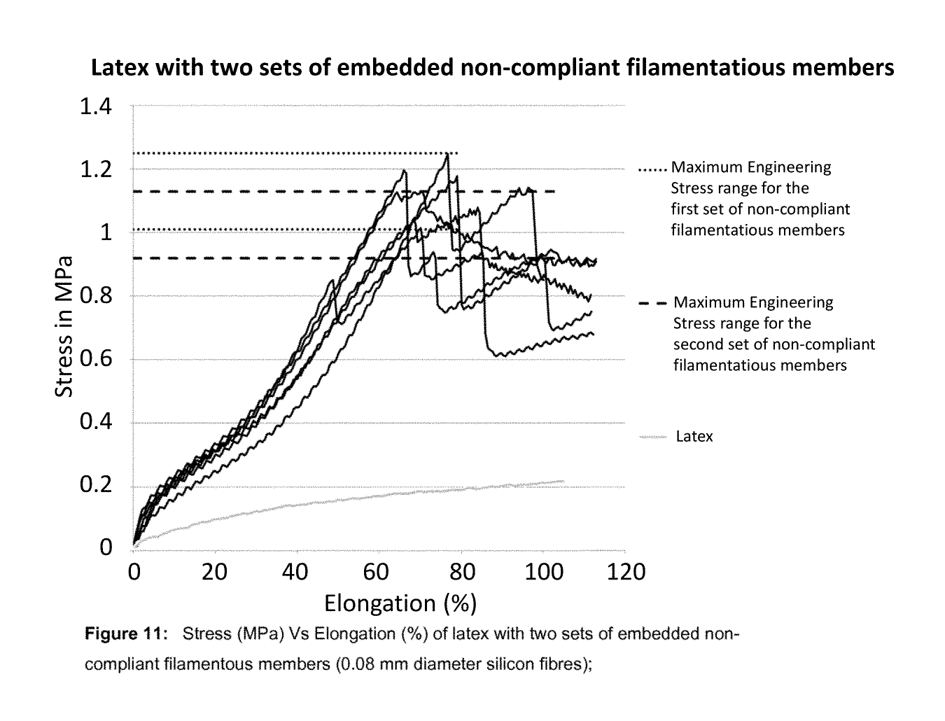

FIG. 11 shows latex samples embedded with two sets of non-compliant filamentous members undergoing uniaxial tensile testing. Each sample demonstrates the initial fracture of the first set of non-compliant filamentous members embedded in the latex, the second set of non-compliant filamentous member taking up the load and their subsequent failure. The maximum engineering stress range is highlighted by the dashed lines for the failure of first set of non-compliant filamentous members and the second set of non-compliant filamentous members. A comparison to natural latex is shown at the bottom of the graph to highlight the different properties in the new combination material.

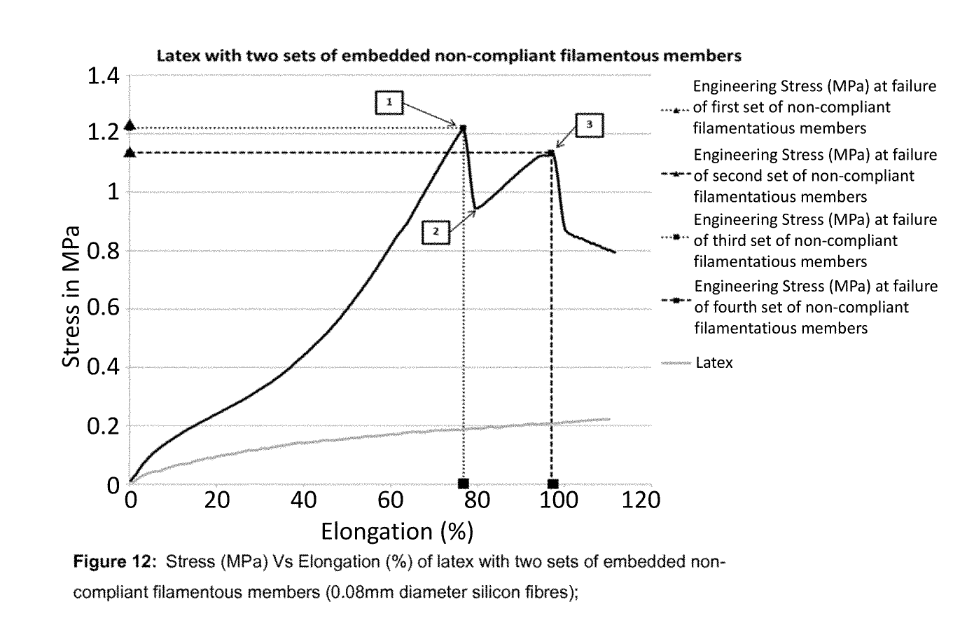

FIG. 12 looks at a single latex sample with two sets of non-compliant filamentous members embedded in it. This shows clearly how the first set of non-compliant filamentous members controls the elongation of the sample until it fails at marker 1. The stress drops to marker 2. This drop is due to the failure of the first set of non-compliant filamentous members which no longer bears the load and the load is them supported by the second set of non-compliant filamentous members and the latex. The second set of non-compliant filamentous members then dictates the elongation of the sample until marker 3. Marker 3 shows the failure of the second set of non-compliant filamentous member. The stress in the sample decreases further despite the elongation increase because both sets of non-compliant filamentous members have failed.

The key finding obtained from FIG. 12, is the controlled expansion of the sample up to failure of the first set of non-compliant filamentous members and the subsequent partitioning of stress to the second set of non-compliant filamentous members. The first set of non-compliant filamentous members fails at a greater stress than the first set of non-compliant filamentous members shown in FIG. 7. This can be attributed to the increased number of non-compliant filamentous members in the material which bear the load. The second set of non-compliant filamentous members fails in the same range shown in Table 2 for the single set of non-compliant filamentous members samples.

SEM Analysis of Latex Layers.

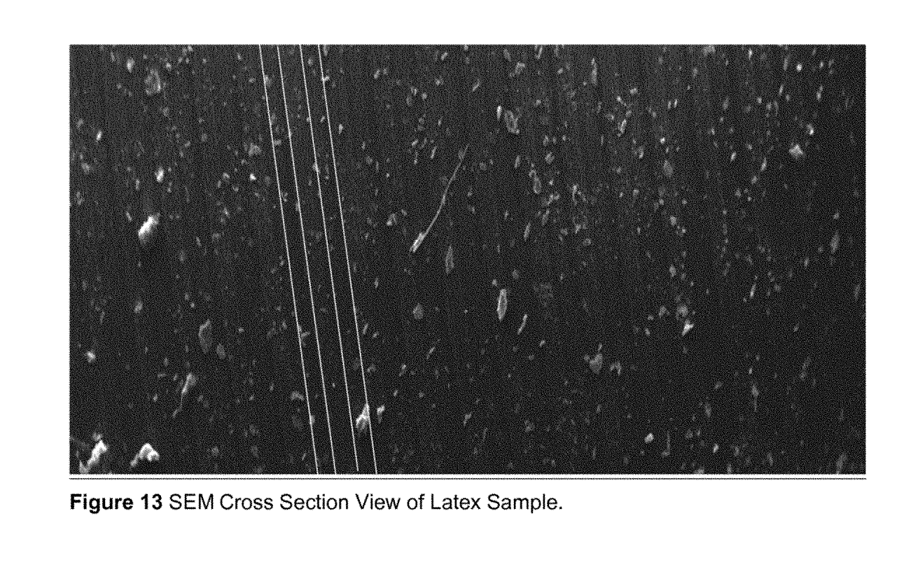

Latex samples were created using gravity casting to create a multi-layer section whereby each layer dried, a new layer was added. This method was repeated until the desired thickness was obtained. This resulted in boundary layer formation in the material. A cross section of the sample has been taken and imaged using a SEM, see FIG. 13. FIG. 13 is a cross section view of the latex sample. No defects between the boundary layers can be seen across multiple samples. The lines highlight three layers of the sample.

Discussion

Latex is a naturally occurring compliant material used in medical devices globally. In Table 1, the tensile test data for latex can be seen. The results identify latex's natural compliance and ability to stretch up to 9.72 times its initial length when loaded to a stress of 1.41 MPa. This low stiffness is one of the main reasons why latex is used in compliant balloons for medical procedures. With a relatively low luminal pressure, a latex balloon can be expanded to block an artery for a short time to allow a procedure to be completed. However, there is no control over the inflation of the balloon once luminal pressure is introduced. Given the highly compliant nature of latex, the pressurised balloon will take up the shape of its surroundings. This is effective for obstruction but is not effective for controlled expansion and hence latex balloons are generally not used for device delivery.

When two materials are combined, the resulting material has a mixture of material properties from both of the constituent materials. Table 3 shows the results of tensile testing samples made from a latex matrix embedded with silicon fibres, referred to as a set of non-compliant filamentous members. The results show samples with a higher stiffness compared to latex alone, i.e. lower elongation for a given stress value. This demonstrates how embedded non-compliant filamentous members can control the elongation of the samples when load is applied. If this is considered in the context of medical balloons, for example latex balloons with embedded non-compliant filamentous members, then the pattern of undulation and length of the embedded non-compliant filamentous members can be used to control the expansion of the balloon to predictable sizes/diameters.

Table 4 shows the results from tensile testing latex samples with two sets of non-compliant filamentous members/fibres embedded. The table shows the first elongation of the sample to a stress range between 1.01-1.25 MPa with an elongation between 67-79% of initial length (shown by the dashed section in FIG. 9.). As further stress was applied to the samples, a second peak developed (shown by the dashed section in FIG. 9) which illustrates how the load was picked up by the second set of non-compliant filamentous members and the elongation of the sample controlled. The second set of non-compliant filamentous members/fibres failed in the same manner as the first and allowed for further elongation and strain hardening of the test samples. During the non-compliant filamentous members failure, no damage was observed to the latex matrix material.

Applying these findings to a medical balloon, a balloon with multiple sets of non-compliant filamentous members would be a multiple size/diameter balloon and its expansion would be predictable and controllable. This balloon could have determined sizes/diameters depending on the undulation pattern and number of sets of non-compliant filamentous members embedded in the matrix material of the balloon. A balloon having such properties is exemplified below.

Formation of Cylindrical Balloons.

In a manner analogous to the samples above and using the same materials a balloon was produced by dip coating a cylindrical mandrel with latex (same latex material as above) (4 coatings), by placing undulated silicon-PTFE tempered filamentous members/fibres (of the type described above) along the length of the balloon and dip coating the balloon in latex (same latex material as above) for a further 4 layers. Two different groups of fibres were used with two different extensible lengths giving rise to the two expansion diameters of roughly 5 mm and 8 mm. This resulted in cylindrical balloons being formed using the latex material and with embedded fibres. The balloons have embedded non-compliant filamentous members in a compliant matrix such that the first set of filamentous members extended and constrained the balloon to a first inflation diameter (5 mm) as shown in FIG. 14(a) and on further pressurisation these fibres failed and the second inflation diameter of the balloon was reached (8 mm) constrained by the second group of fibres as shown in FIG. 14(b). In these figures a callipers is provided for the purposes of showing the dimensions.

Conclusion

In this study, latex samples have been produced with multiple sets of non-compliant filamentous members embedded. Through tensile testing it has been demonstrated how the undulation pattern of the non-compliant filamentous members/fibres can determine the elongation of the sample when stress is applied. Highlighted in the results is the repeatability of the elongation versus stress relationship throughout the tests. In addition, increasing the number of non-compliant filamentous members/fibres embedded in the matrix increases the initial stress to failure for the first set of non-compliant filamentous members and the subsequent sets of non-compliant filamentous members have a decreasing trend in stress at failure although elongation is markedly increased.

Using the results from this study, it is clear that a balloon with embedded non-compliant filamentous members can be created to have multiple predetermined sizes/diameters depending on the number (and undulation patterns) of the embedded sets of non-compliant filamentous members. These non-compliant filamentous members may also be used to change the expanded shape of the balloon. No other angioplasty/valvuloplasty balloon is capable of this, which presents a clear advantage of the present invention.

Methods:

Sets of Non-Compliant Filamentous Members

Monofilament fibres are the non-compliant filamentous members used to control the expansion of the samples. The non-compliant filamentous members are crimped into predetermined undulation patterns. This allows the non-compliant filamentous members to be assigned to different groups or ‘sets depending on their undulation pattern. Each set of non-compliant filamentous members represents a group of non-compliant filamentous members which are the same, namely they are made from the same material, and have the same thickness and the same degree of undulation. Each embedded set of non-compliant filamentous members represents a set size/diameter that the sample can predictably achieve.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. An inflation balloon comprising:
(a) a compliant balloon wall;
(b) a first set of non-compliant filamentous members within the balloon wall for restraining the balloon to a first expanded size,
(c) a second set of non-compliant filamentous members within the balloon wall for restraining the balloon to a second larger expanded size,
wherein the first set of non-compliant filamentous members are arranged to undergo failure to allow the balloon to expand from the first expanded size to the second larger expanded size.

2. An inflation balloon according to claim 1 wherein the first set of non-compliant filamentous members are arranged to be slack so as to allow inflation of the inflation balloon, but to restrain the balloon to an expanded diameter when all of the slack has been taken up by expansion of the balloon.

3. An inflation balloon according to claim 1 wherein the second set of non-compliant filamentous members are arranged to be slack so as to allow inflation of the inflation balloon, but to restrain the balloon to an expanded diameter when all of the slack has been taken up by expansion of the balloon.

4. An inflation balloon according to claim 1 wherein the first set of non-compliant filamentous members and the second set of non-compliant filamentous members are arranged so that the balloon when inflated has different parts with different diameters.

5. An inflation balloon according to claim 1 wherein the first set of non-compliant filamentous members and the second set of non-compliant filamentous members differ from each other by being of different materials, different lengths, or different thickness or any combination thereof

6. An inflation balloon according to claim 1 wherein the first or second set of non-compliant filamentous members follow a non-linear path around a circumference of the balloon or both the first and second set of non-compliant filamentous members follow a non-linear path around a circumference of the balloon.

7. An inflation balloon according to claim 1 wherein the first or second set of non-compliant filamentous members follow a wave-like path around a circumference of the balloon, or both first and second set of non-compliant filamentous members follow a wave-like path around a circumference of the balloon.

8. An inflation balloon according to claim 1 wherein the first set of non-compliant filamentous members are in the form of rings that run about a circumference of the balloon.

9. An inflation balloon according to claim 1 wherein the second set of non-compliant filamentous members are in the form of rings that run about a circumference of the balloon.

10. An inflation balloon according to claim 1 wherein the first or second set of non-compliant filamentous members are monofilament materials such as monofilament fibres or both the first and second set of non-compliant filamentous members are monofilament materials such as monofilament fibres.

11. A medical device such as a balloon catheter device comprising a substrate, such as a catheter, on which a balloon as defined claim 1 is mounted.

12. A method of inflating an inflation balloon according to claim 1 comprising: providing an inflation balloon according to claim 1, inflating the inflation balloon to a first expansion size and subsequently using inflation pressure to cause the first set of non-compliant filamentous members to undergo failure to allow the balloon to expand to a second larger expanded size.

13. A method of operation of a device, comprising providing a device according to claim 11, inserting it into the human or animal body, inflating the inflation balloon to a first expansion size and subsequently using inflation pressure to cause the first set of non-compliant filamentous members to undergo failure to allow the balloon to expand to a second larger expanded size.
